(19) United States
(12) Patent Application Publication
(10) Pub. No.: US 2007/0208031 A1
(43) Pub. Date: Sep. 6, 2007

(54) Substituted Diketopiperazines as Oxytocin Antagonists

(76) Inventors: Alan David Borthwick, Stevenage (GB); Steven Leslie Sollis, Stevenage (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/630,097

(22) PCT Filed: Jun. 21, 2005
(86) PCT No.: PCT/EP05/06761

§ 371 Date: Dec. 19, 2006

(30) Foreign Application Priority Data
Jun. 23, 2004 (GB) 0414092.7

(51) Int. Cl. A61K 31/496; C07D 403/02

(52) U.S. Cl. 514/254.06; 544/371

(57) ABSTRACT

Compounds of formula (IA)

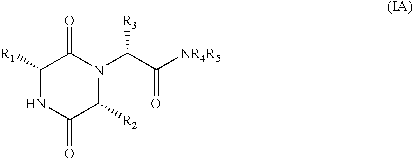

wherein R₁ is 2-indanyl, R₂ is 1-methylpropyl, R₃ is 1-methyl-indazol-5-yl, R₄ represents methyl and R₅ represents hydrogen or methyl, and pharmaceutically acceptable derivatives thereof are described, as are processes for their preparation, pharmaceutical compositions containing them and their use in medicine, particularly their use as oxytocin antagonists.

This invention relates to novel diketopiperazine derivatives having a potent and selective antagonist action at the oxytocin receptor, to processes for their preparation, pharmaceutical compositions containing them and to their use in medicine.

The hormone oxytocin is a potent contractor of the uterus and is used for the induction or augmentation of labour. Also the density of uterine oxytocin receptors increases significantly by >100 fold during pregnancy and peaks in labour (pre-term and term).

Pre-term births/labour (between 24 and 37 weeks) causes about 60% of infant mortality/morbidity and thus a compound which inhibits the uterine actions of oxytocin e.g. oxytocin antagonists, should be useful for the prevention or control of pre-term labour.

International patent application WO 99/47549 describes diketopiperazine derivatives including 3-benzyl-2,5-diketopiperazine derivatives as inhibitors of fructose 1,6-bisphosphate (FBPase).

International patent application WO 03/053443 describes a class of diketopiperazine derivatives which exhibit a particularly useful level of activity as selective antagonists at the oxytocin receptor. A preferred class of compounds described therein is represented by the formula (A).

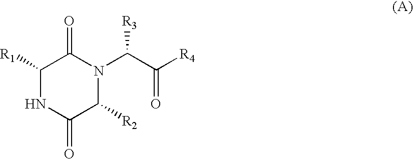

Such compounds include those wherein inter alia R₁ is 2-indanyl, R₂ is C₃₋₄alkyl, R₃ is an optionally substituted 6,5 fused bicyclic ring e.g. 1H-indazol-5-yl linked to the rest of the molecule via a carbon atom in the ring, R₄ represents the group NR₅R₆ wherein R₅ and R₆ each represent alkyl e.g. methyl or R₅ and R₆ together with the nitrogen atom to which they are attached form a 3 to 7 membered saturated heterocyclic ring which heterocycle may contain an additional heteroatom selected from oxygen.

International patent application WO 2005/000840 describes diketopiperazine derivatives of formula (B) 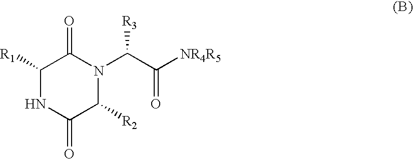
wherein R₁ is 2-indanyl, R₂ is 1-methylpropyl, R₃ is 2-methyl-1,3-oxazol-4-yl and R₄ and R₅ together with the nitrogen atom to which they are attached represent morpholino.

We have now found a novel group of selective oxytocin receptor antagonists which exhibit a particularly advantageous pharmacokinetic profile.

The present invention thus provides at least one chemical entity selected from compounds of formula (I) 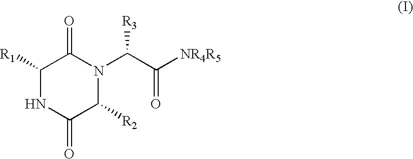
wherein R₁ is 2-indanyl, R₂ is 1-methylpropyl, R₃ is 1-methyl-indazol-5-yl, R₄ represents methyl and R₅ represents hydrogen and pharmaceutically acceptable derivatives thereof.

Alternatively, the present invention provides at least one chemical entity selected from compounds of formula (IA) 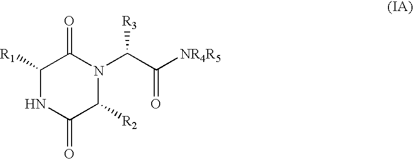
wherein R₁ is 2-indanyl, R₂ is 1-methylpropyl, R₃ is 1-methyl-indazol-5-yl, R₄ represents methyl and R₅ represents hydrogen or methyl, and pharmaceutically acceptable derivatives thereof.

It will be appreciated that the compounds of formula (I) and formula (IA) possess the absolute stereochemistry depicted at the asymmetric carbon atoms bearing groups R₁, R₂ and R₃, ie the stereochemistry at these positions is always (R). Nevertheless, it should also be appreciated that although such compounds are substantially free of the (S)-epimer at each of R₁, R₂ and R₃, each epimer may be present in small amounts, for example 1% or less of the (S)-epimer may be present.

It will also be appreciated that the group R₂ contains an asymmetric carbon atom and that the invention includes both the (R)- and (S)-epimers thereof.

In one embodiment of the invention, R₂ is (1S)-1-methylpropyl. In another embodiment of the invention, R₂ is (1R)-1-methylpropyl.

In one embodiment of the invention, R₅ represents hydrogen. In another embodiment of the invention, R₅ represents methyl.

In one embodiment of the invention is the compound the preparation of which is specifically described in example 1. In another embodiment of the invention is the compounds the preparation of which is specifically described in examples 1 and 2.

In one aspect, chemical entities useful in the present invention may be at least one chemical entity selected from:

(2R)-2-{(3R,6R)-3-(2,3-dihydro-1H-inden-2-yl)-6-[(1S)-1-methylpropyl]-2,5-dioxo-1-piperazinyl}-N-methyl-2-(1-methyl-1H-indazol-5-yl)ethanamide, and

(2R)-2-{(3R,6R)-3-(2,3-dihydro-1H-inden-2-yl)-6-[(1S)-1-methylpropyl]-2,5-dioxo-1-piperazinyl}-N,N-dimethyl-2-(1-methyl-1H-indazol-5-yl)ethanamide, and pharmaceutically acceptable derivatives thereof.

As used herein, the term “pharmaceutically acceptable” means a compound which is suitable for pharmaceutical use. Salts and solvates of compounds of the invention which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of the invention and their pharmaceutically acceptable salts and solvates.

As used herein, the term “pharmaceutically acceptable derivative”, means any pharmaceutically acceptable salt, solvate, or prodrug e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol 1: Principles and Practice, which is incorporated herein by reference to the extent of teaching such derivatives. In one aspect, pharmaceutically acceptable derivatives are salts, solvates, esters, carbamates and phosphate esters. In another aspect, pharmaceutically acceptable derivatives are salts, solvates and esters. In one aspect, pharmaceutically acceptable derivatives are physiologically acceptable salts. In a further aspect, pharmaceutically acceptable derivatives are solvates and esters. In another aspect, pharmaceutically acceptable derivatives are solvates.

Suitable physiologically acceptable salts of compounds of the present invention include acid addition salts formed with physiologically acceptable inorganic acids or organic acids. Examples of such acids include hydrochloric acid, hydrobromic acid, nitric acid, phosphoric acid, sulphuric acid, sulphonic acids e.g. methanesulphonic, ethanesulphonic, benzenesulphonic and p-toluenesulphonic, citric acid, tartaric acid, lactic acid, pyruvic acid, acetic acid, succinic acid, fumaric acid and maleic acid.

The present invention also relates to solvates of the compounds of formula (I) or formula (IA), for example hydrates, or solvates with pharmaceutically acceptable solvents including, but not limited to, alcohols, for example ethanol, iso-propanol, acetone, ethers, esters, e.g. ethyl acetate.

The compounds of the invention may also be used in combination with other therapeutic agents. The invention thus provides, in a further aspect, a combination comprising a compound of the invention or a pharmaceutically acceptable derivative thereof together with a further therapeutic agent.

When a compound of the invention or a pharmaceutically acceptable derivative thereof is used in combination with a second therapeutic agent active against the same disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may be used in combination with tocolytics or prophylactic medicines. These include, but are not limited to, beta-agonists such as terbutaline or ritodrine, calcium channel blockers, e.g. nifedepine, non-steroidal anti-inflammatory drugs, such as indomethacin, salts of magnesium, such as magnesium sulphate, other oxytocin antagonists, such as atosiban, and progesterone agonists and formulations. In addition the compounds of the present invention may be used in combination with antenatal steroids including betamethasone and dexamethasone, prenatal vitamins especially folate supplements, antibiotics, including but not limited to ampicillin, amoxicillin/clavulanate, metronidazole, clindamycin, and anxiolytics.

In one aspect, the combinations referred to above may be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The compounds of formula (I) and formula (IA) have a high affinity for the oxytocin receptors on the uterus of rats and humans and this may be determined using conventional procedures. For example the affinity for the oxytocin receptors on the rat uterus may be determined by the procedure of Pettibone et al, Drug Development Research 30. 129-142 (1993). The compounds of the invention also exhibit high affinity at the human recombinant oxytocin receptor in CHO cells and this may be conveniently demonstrated using the procedure described by Wyatt et al. Bioorganic & Medicinal Chemistry Letters, 2001 (11) p 1301-1305.

The compounds of the invention exhibit an advantageous pharmacokinetic profile including good bioavailability and low intrinsic clearance. In one aspect, the compounds of the invention exhibit good potency and low intrinsic clearance. In another aspect, the compounds of the invention exhibit low intrinsic clearance.

The compounds of the invention are therefore useful in the treatment or prevention of diseases and/or conditions mediated through the action of oxytocin. Examples of such diseases and/or conditions include pre-term labour, dysmenorrhea, endometriosis and benign prostatic hyperplasia.

The compounds may also be useful to delay labour prior to elective caesarean section or transfer of the patient to a tertiary care centre, treatment of sexual dysfunction (male and female), particularly premature ejaculation, obesity, eating disorders, congestive heart failure, arterial hypertension, liver cirrhosis, nephritic or ocular hypertension, obsessive-compulsive disorder and neuropsychiatric disorders. The compounds of the invention may also be useful for improving fertility rates in animals, e.g. farm animals.

The invention therefore provides for at least one chemical entity selected from compounds of formula (I) or formula (IA) and pharmaceutically acceptable derivatives thereof for use in therapy, particularly for use in human or veterinary therapy, and in particular for use as a medicine for antagonising the effects of oxytocin upon the oxytocin receptor.

The invention also provides for the use of at least one chemical entity selected from compounds of formula (I) or formula (IA) and pharmaceutically acceptable derivatives thereof for the manufacture of a medicament for antagonising the effects of oxytocin on the oxytocin receptor.

According to a further aspect, the invention also provides for a method for antagonising the effects of oxytocin upon the oxytocin receptor, comprising administering to a patient in need thereof an antagonistic amount of at least one chemical entity selected from compounds of formula (I) or formula (IA) and pharmaceutically acceptable derivatives thereof.

It will be appreciated by those skilled in the art that reference herein to treatment extends to prophylaxis as well as the treatment of established diseases or symptoms.

It will further be appreciated that the amount of a compound of the invention required for use in treatment will vary with the nature of the condition being treated, the route of administration and the age and the condition of the patient and will be ultimately at the discretion of the attendant physician. In general however doses employed for adult human treatment will typically be in the range of 2 to 1000 mg per day, dependent upon the route of administration.

Thus for parenteral administration a daily dose will typically be in the range 2 to 50 mg, in one aspect 5 to 25 mg per day. For oral administration a daily dose will typically be within the range 10 to 1000 mg, e.g. 50 to 500 mg per day.

The desired dose may be presented in a single dose or as divided doses administered at appropriate intervals, for example as two, three, four or more sub-doses per day.

While it is possible that, for use in therapy, a compound of the invention may be administered as the raw chemical, it is preferable to present the active ingredient as a pharmaceutical formulation.

The invention thus further provides a pharmaceutical formulation comprising at least one chemical entity selected from compounds of formula (I) or formula (IA) and pharmaceutically acceptable derivatives thereof together with one or more pharmaceutically acceptable carriers thereof and, optionally, other therapeutic and/or prophylactic ingredients. The carrier(s) must be ‘acceptable’ in the sense of being compatible with the other ingredients of the formulation and not deleterious to the recipient thereof.

The compositions of the invention include those in a form especially formulated for oral, buccal, parenteral, inhalation or insufflation, implant, vaginal or rectal administration.

Tablets and capsules for oral administration may contain conventional excipients such as binding agents, for example, syrup, acacia, gelatin, sorbitol, tragacanth, mucilage of starch or polyvinylpyrrolidone; fillers, for example, lactose, sugar, microcrystalline cellulose, maize-starch, calcium phosphate or sorbitol; lubricants, for example, magnesium stearate, stearic acid, talc, polyethylene glycol or silica; disintegrants, for example, potato starch or sodium starch glycollate, or wetting agents such as sodium lauryl sulphate. The tablets may be coated according to methods well known in the art. Oral liquid preparations may be in the form of, for example, aqueous or oily suspensions, solutions emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxyethylcellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example, almond oil, fractionated coconut oil, oily esters, propylene glycol or ethyl alcohol; solubilizers such as surfactants for example polysorbates or other agents such as cyclodextrins; and preservatives, for example, methyl or propyl p-hydroxybenzoates or ascorbic acid. The compositions may also be formulated as suppositories, e.g. containing conventional suppository bases such as cocoa butter or other glycerides.

For buccal administration the composition may take the form of tablets or lozenges formulated in conventional manner.

The composition according to the invention may be formulated for parenteral administration by injection or continuous infusion. Formulations for injection may be presented in unit dose form in ampoules, or in multi-dose containers with an added preservative. The compositions may take such forms as suspensions, solutions, or emulsions in oily or aqueous vehicles, and may contain formulatory agents such as suspending, stabilising and/or dispersing agents. Alternatively the active ingredient may be in powder form for constitution with a suitable vehicle, e.g. sterile, pyrogen-free water, before use.

The compositions according to the invention may contain between 0.1-99% of the active ingredient, conveniently from 1-50% for tablets and capsules and 3-50% for liquid preparations.

The advantageous pharmacokinetic profile of the compounds of the invention is readily demonstrated using conventional procedures for measuring the pharmacokinetic properties of biologically active compounds.

The compounds of the invention and pharmaceutically acceptable derivatives thereof may be prepared by the processes described hereinafter, said processes constituting a further aspect of the invention. In the following description, the groups are as defined above for compounds of the invention unless otherwise stated.

Thus, compounds of formula (I) or formula (IA) may be prepared by reaction of the carboxylic acid (II), wherein R₁, R₂ and R₃ have the meanings defined in formula (I) and formula (IA), and the chirality at R₃ is either R or S, or a mixture thereof, 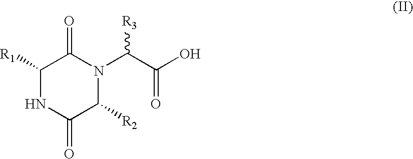
or an activated derivative thereof with the amine HNR₄R₅ wherein R₄ and R₅ have the meaning defined in formula (I) and formula (IA) under standard conditions for preparing amides from a carboxylic acid or an activated derivative thereof and an amine.

It will be appreciated that the mixture of diastereomers of compounds of formula (I) or formula (IA) obtained from the above reaction may be separated using standard resolution techniques well known in the art, for example column chromatography.

Thus the amide of formula (I) or formula (IA) may be prepared by treating the carboxylic acid of formula (II) with an activating agent such as BOP (benzotriazol-1-yloxy-tris(dimethylamino)phosphonium hexafluorophosphate), TBTU (2-(1H-benzotriazol-1-yl)-1,1,3,3-tetramethyluronium tetrafluoroborate), BOP-Cl (bis(2-oxo-3-oxazolidinyl)phosphinic chloride), oxalyl chloride or 1,1′-carbonyldiimidazole in an aprotic solvent such as dichloromethane optionally in the presence of a tertiary amine such as triethylamine and subsequent reaction of the product thus formed, ie the activated derivative of the compound of formula (II), with the amine HNR₄R₅.

Alternatively the amide of formula (I) or formula (IA) may be prepared by reacting a mixed anhydride derived from the carboxylic acid (II) with the amine HNR₄R₅ in an aprotic solvent such as tetrahydrofuran. Conveniently the reaction is carried out at low temperatures, for example 25° C. to −90° C., more conveniently at approximately −78° C.

The mixed anhydride is conveniently prepared by reacting the carboxylic acid (II) with a suitable acid chloride e.g. pivalolyl chloride in an aprotic solvent such as ethyl acetate in the presence of a tertiary organic base such as a trialkylamine e.g. triethylamine and at low temperatures, for example 25° C. to −90° C., conveniently at approximately −78° C.

Compounds of formula (I) or formula (IA) may also be prepared by reacting a compound of formula (III) 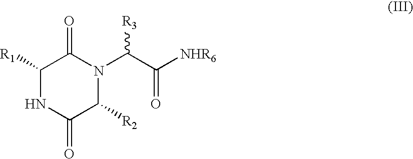
wherein R₁, R₂ and R₃ have the meanings defined in formula (I) and formula (IA) and R₆ is 2-hydroxyphenyl, with 1,1′-carbonyldiimidazole or 1,1′-thiocarbonyidiimidazole in a suitable solvent such as dichloromethane and subsequent reaction of the products thus formed with the amine HNR₄R₅.

Compounds of formula (II) may be prepared from a compound of formula (III) wherein R₆ is 2-hydroxyphenyl by reaction with 1,1′-carbonyldiimidazole or 1,1′-thiocarbonyldiimidazole in a suitable solvent such as dichloromethane and subsequent reaction of the product thus formed with aqueous acetone.

Compounds of formula (III) wherein R₆ is 2-hydroxyphenyl may be prepared from the corresponding compounds of formula (III) wherein R₆ is a 2-benzyloxyphenyl group by hydrogenolysis using hydrogen and a palladium catalyst.

Compounds of formula (III) wherein R₆ is a 2-benzyloxyphenyl group may be prepared from a compound of formula (IV) 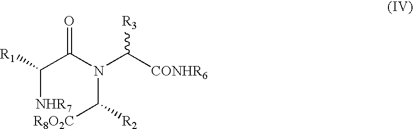
wherein R₁, R₂ and R₃ have the meanings defined in formula (I) and formula (IA), R₆ is 2-benzyloxyphenyl, R₇ is t-butyloxycarbonyl and R₈ is C₁₋₆alkyl by reaction with hydrogen chloride in a solvent such as dioxan, followed by treatment with a base such as triethylamine in methanol.

Compounds of formula (IV) may be prepared by reacting the amino ester hydrochloride (V), 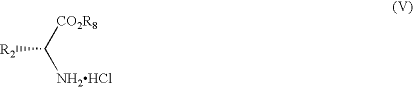
wherein R₁ has the meanings defined in formula (I) and formula (IA) and R₈ is C₁₋₆alkyl, with an aldehyde R₃CHO (VI), wherein R₃ has the meaning defined in formula (I) and formula (IA), in the presence of triethylamine and in a solvent such as trifluoroethanol and then reacting the resultant product with a compound of formula (VII) 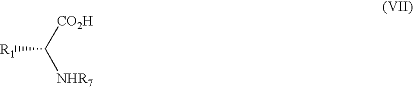
wherein R₁ has the meaning defined in formula (I) and formula (IA), and R₇ is t-butyloxycarbonyl or benzyloxycarbonyl and the isocyanide CNR₆ (VIII) wherein R₆ is a 2-benzyloxyphenyl group, in a solvent such as trifluoroethanol.

Compounds of formula (III) wherein R₆ is a 2-benzyloxyphenyl group may be prepared from a compound of formula (IV) wherein R₁, R₂ and R₃ have the meanings defined in formula (I) and formula (IA), R₆ is 2-benzyloxyphenyl and R₇ is t-butyloxycarbonyl by the reaction with hydrogen chloride in dioxan followed with triethylamine in a solvent such as dichloromethane.

The compound of formula (IV) wherein R₇ is t-butyloxycarbonyl may be prepared by the route described above using a compound of formula (VII) wherein R₇ is t-butyloxycarbonyl.

The R₂ substituent is a 1-methylpropyl group and the compound of formula (I) and formula (IA) wherein R₂ is a 1-methylpropyl group having an (S) or (R) configuration may be prepared by starting with the aminoester hydrochloride (V) wherein the R₂ group has the required (S) or (R) configuration.

Aminoester hydrochloride (V), wherein R₁ has the meaning defined in formula (I) and formula (IA) and R₈ is C₁₋₆ alkyl, may be prepared from the corresponding commercially available amino acids, D-alloisoleucine or D-isoleucine, by the method of Schmidt, U; Kroner, M; Griesser, H. Synthesis (1989), (11), 832-5.

Aldehyde R₃CHO (VI), wherein R₃ has the meaning defined in formula (I) and formula (IA), may be prepared from the commercially available bromo compound R₃Br, wherein R₃ has the meaning defined in formula (I) and formula (IA), by the method of V. Auwers; Lange; Chem. Ber.; 55; 1922; 1141, 1157. Alternatively, aldehyde R₃CHO (VI) may be prepared from the commercially available nitrile compound R₃CN, wherein R₃ has the meaning defined in formula (I) and formula (IA), by the method of Halley, Frank; Sava, Xavier. Synthesis of 5-cyanoindazole and 1-methyl and 1-aryl-5-cyanoindazoles. Synthetic Communications (1997), 27(7), 1199-1207.

The aminoacid derivative (VII) wherein R₁ has the meaning defined in formula (I) and formula (IA) and R₇ is t-butyloxycarbonyl is commercially available; the aminoacid derivative (VII) wherein R₁ has the meaning defined in formula (I) and formula (IA) and R₇ is benzyloxycarbonyl may be prepared from the corresponding commercially available amino acid (R)-R₁CH(NH₂)CO₂H (IX), wherein R₁ has the meaning defined in formula (I) and formula (IA), by treatment with N-(benzyloxycarbonyloxy)succinimde and triethylamine in a solvent such as dioxane in water.

The isocyanide CNR₆ (VIII) may be prepared according to literature methods (Obrecht, Roland; Herrmann, Rudolf; Ugi, Ivar, Synthesis, 1985, 4, 400-402).

Acid addition salts of the compound of formula (I) and formula (IA) may be prepared by conventional means, for example, by treating a solution of the compound in a suitable solvent such as dichloromethane or acetone, with a suitable solution of the appropriate inorganic or organic acid.

The following examples are illustrative, but not limiting of the embodiments of the present invention.

Experimental

Abbreviations

DIBAL—diisobutylaluminium chloride

Nomenclature

All intermediates and examples were named using ACD Name Pro 6.02 in ISISDraw.

General Purification and Analytical Methods

Analytical HPLC was conducted on a Supelcosil LCABZ+PLUS column (3.3 cm×4.6 mm ID), eluting with 0.1% HCO₂H and 0.01 M ammonium acetate in water (solvent A), and 0.05% HCO₂H and 5% water in acetonitrile (solvent B), using the either elution gradient 1, 0-0.7 minutes 0% B, 0.7-4.2 minutes 0%-100% B, 4.2-5.3 minutes 100% B, 5.3-5.5 minutes 0% B or elution gradient 2, 0-0.7 minutes 0% B, 0.7-4.2 minutes 0%-100% B, 4.2-4.6 minutes 100% B, 4.6-4.8 minutes 0% B at a flow rate of 3 ml/minute. Retention times (Rt) are quoted in minutes. The mass spectra (MS) were recorded on a Waters ZQ 2000 mass spectrometer using electrospray positive [ES+ve to give MH⁺ and M(NH₄)⁺ molecular ions] or electrospray negative [ES-ve to give (M-H)⁻ molecular ion] modes. ¹H NMR spectra were recorded using a Bruker DPX 400 MHz spectrometer using tetramethylsilane as the external standard.

Purification using silica cartridges refers to chromatography carried out using a Combiflash® Companion™ with Redisep® cartridges supplied by Presearch. Hydrophobic frits refer to filtration tubes sold by Whatman. SPE (solid phase extraction) refers to the use of cartridges sold by International Sorbent Technology Ltd. TLC (thin layer chromatography) refers to the use of TLC plates sold by Merck coated with silica gel 60 F₂₅₄.

Intermediate 1 (Method A)

1-Methyl-1H-indazole-5-carbaldehyde

A 2.0M solution of n-butyl magnesium chloride in tetrahydrofuran (3.05 ml) was added to toluene (20 ml) under nitrogen and cooled to −10° C. To this was added a 1.6M solution of n-butyl lithium in hexanes (7.63 ml) and after 1 hour the reaction mixture was cooled to −30° C. To this was added a solution of 5-bromo-1-methyl-1H-indazole¹ (2.35 g) in tetrahydrofuran (10 ml) and the reaction mixture was warmed to −10° C. After 1 hour dimethylformamide (5 ml) was added and the reaction mixture was stirred at −10° C. for 1 hour. The reaction was quenched using 2N hydrochloric acid (20 ml) and the reaction allowed to warm to room temperature. After 30 minutes the reaction mixture was basified with saturated aqueous sodium bicarbonate solution and then extracted using ethyl acetate (2×80 ml). The organic phase was washed with sodium bicarbonate solution (2×100 ml) and then 10% lithium chloride in water (2×100 ml) and then brine. The organic phase was dried over anhydrous magnesium sulphate and evaporated in vacuo. The residue was applied to a silica Redisep® cartridge (120 g) and eluted with 10-30% ethyl acetate in cyclohexane. The required fractions were combined and evaporated in vacuo to give 1-methyl-1H-indazole-5-carbaldehyde (1.43 g, 80%) as a white solid.
HPLC Rt=2.2 minutes (gradient 1); m/z [M+H]⁺=161 (gradient 1)
Intermediate 1 (Method B)
1 -Methyl-1H-indazole-5-carbaldehyde

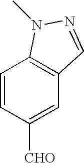

To a solution of 1-methyl-1H-indazole-5-carbonitrile² (7 g) in anhydrous toluene (300 ml) under nitrogen at −70° C. was added a 1.5 M solution of DIBAL in toluene (59.4 ml) drop wise over approx 20 minutes. The reaction mixture was allowed to warm to −60° C. and stirred at that temperature for 4 hours, the cooling bath removed and then quenched by drop wise addition of acetic acid (30 ml) (care evolution of gas). Water (240 ml) was added and mixture vigorously stirred for 30 minutes and then extracted with ethyl acetate (200 ml). The organic phase was washed with water (100 ml) and then brine (100 ml) dried over anhydrous magnesium sulphate, filtered and concentrated in vacuo to give 1-methyl-1H-indazole-5-carbaldehyde (6.8 g, 95%) as a pale yellow solid, consistent in all respects with that obtained from 5-bromo-1-methyl-1H-indazole obtained above.
Intermediate 2 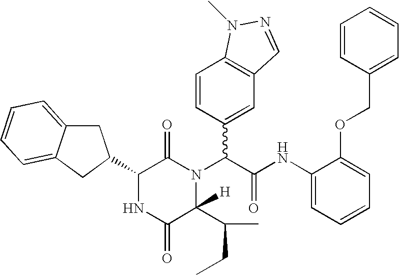
2-{(3R,6R)-3-(2,3-Dihydro-1H-inden-2-yl)-6-[(1S)-1-methylpropyl]-2,5-dioxo-1-piperazinyll}-2-(1-methyl-1H-indazol-5-yl)-N-{2-[(phenylmethyl)oxy]phenyl}acetamide

1-Methyl-1H-indazole-5-carbaldehyde (intermediate 1) (1.66 g) and methyl D-alloisoleucinate hydrochloride (1.88 g) were dissolved in 2,2,2-trifluoroethanol (30 ml) and methanol (30 ml). To this was added triethylamine (1.44 ml) and the reaction mixture stirred at room temperature under N₂ for 3.5 hours. (2R)-2,3-Dihydro-1H-inden-2-yl({[(1,1-dimethylethyl)oxy]carbonyl}amino)ethanoic acid (3.01 g) and 2-[(phenylmethyl)oxy]phenyl isocyanide (2.16 g) were added to the reaction mixture and the solution was left to stand at room temperature for 3 days. The solvent was removed in vacuo. The residue was dissolved in dichloromethane and evaporated in vacuo. The residue was dissolved in 4N hydrogen chloride in dioxan (20 ml) and the reaction mixture was stirred for 1 hour. The solvent was removed in vacuo and co-evaporated with methanol x3. The residue was dissolved in methanol (70 ml). To this was added triethylamine (6 ml) while the flask stood on dry ice. The reaction mixture was left to stand for 20 hours at room temperature. The solvent was evaporated in vacuo and the residue concentrated from methanol (x1) and dichloromethane (x1). The residue was separated between ethyl acetate and aqueous sodium bicarbonate solution. The organic phase was washed with aqueous sodium bicarbonate solution, water, brine and dried over anhydrous magnesium sulphate. The solvent was removed in vacuo and the residue was applied to a silica cartridge (120 g). This was eluted with 30-70% ethyl acetate in cyclohexane. The required fractions were combined and evaporated in vacuo to give 2-{(3R,6R)-3-(2,3-dihydro-1H-inden-2-yl)-6-[(1S)-1-methylpropyl]-2,5-dioxo-1-piperazinyl}-2-(1-methyl-1H-indazol-5-yl)-N-{2-[(phenylmethyl)oxy]phenyl}acetamide (4.15 g, 62%) as a yellow solid.
HPLC Rt=3.62, 3.66 minutes (gradient 1); m/z [M+H]⁺=656.
Intermediate 3 (Method A) 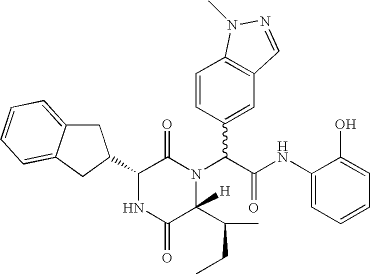
2-{(3R,6R)-3-(2,3-Dihydro-1H-inden-2-yl)-6-[(1S)-1-methylpropyl]-2,5-dioxo-1-piperazinyl}-N-(2-hydroxyphenyl)-2-(1-methyl-1H-indazol-5-yl)acetamide

2-{(3R,6R)-3-(2,3-Dihydro-1H-inden-2-yl)-6-[(1S)-1-methylpropyl]-2,5-dioxo-1-piperazinyl}-2-(1 -methyl-1 H-indazol-5-yl)-N-{2-[(phenylmethyl)oxy]phenyl}acetamide (intermediate 2) (0.20 g) was dissolved in ethanol (20 ml) and hydrogenated over palladium on charcoal (wet 10% Pd, 50 mg) for 20 hours. The catalyst was removed by filtration and washed with ethanol/dichloromethane (1:1 v/v). The combined washings and filtrate were evaporated in vacuo to give 2-{(3R,6R)-3-(2,3-dihydro-1H-inden-2-yl)-6-[(1S)-1-methylpropyl]-2,5-dioxo-1-piperazinyl}-N-(2-hydroxyphenyl)-2-(1-methyl-1H-indazol-5-yl)acetamide (0.19 g, 100%) as a white solid.

Intermediate 3 (Method A)

2-{(3R,6R)-3-(2,3-Dihydro-1H-inden-2-yl)-6-[(1S)-1-methylpropyl}-2,5-dioxo-1-piperazinyll-N-(2-hydroxyphenyl)-2-(1-methyl-1H-indazol-5-yl)acetamide

2-{(3R,6R)-3-(2,3-Dihydro-1H-inden-2-yl)-6-[(1S)-1-methylpropyl]-2,5-dioxo-1-piperazinyl}-2-(1-methyl-1H-indazol-5-yl)-N-{2-[(phenylmethyl)oxy]phenyl}acetamide (3.5 g) was dissolved in ethanol (200 ml) and hydrogenated over palladium on charcoal (wet 10% Pd, 350 mg) for 5 hours. The catalyst was removed by filtration washed and the filtrate concentrated in vacuo. This residue was purified on a Redisep® silica column (120 g) eluted with 50-90% ethyl acetate in cyclohexane, to afford 2-{(3R,6R)-3-(2,3-dihydro-1H-inden-2-yl)-6-[(1S)-1-methylpropyl]-2,5-dioxo-1-piperazinyl}-N-(2-hydroxyphenyl)-2-(1-methyl-1H-indazol-5-yl)acetamide (1.54 g) as a white solid.
HPLC Rt=3.3 minutes (gradient 1); m/z [M+H]⁺=566.
Intermediate 3 (Method B) 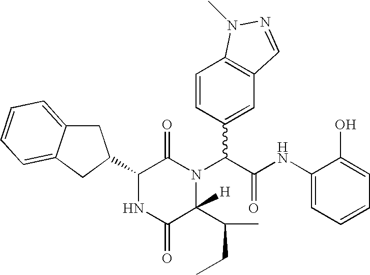
2-{(3R,6R)-3-(2,3-Dihydro-1H-inden-2-yl)-6-[(1S)-1-methylpropyl]-2,5-dioxo-1-piperazinyl}-N-(2-hydroxyphenyl)-2-(1-methyl-1H-indazol-5-yl)acetamide

The methyl N-[(2R)-2-(2,3-dihydro-1H-inden-2-yl)-2-({[(phenylmethyl)oxy]-carbonyl}amino)acetyl]-N-[1-(1 -methyl-1 H-indazol-5-yl)-2-oxo-2-({2-[(phenylmethyl)oxy]phenyl}amino)ethyl]-D-alloisoleucinate (intermediate 4) (22.6 g, 27.5 mmol) was dissolved in ethanol (750 mL and acetic acid (70 mL) and the mixture was hydrogenated at room temperature at 1 atmosphere of H₂ over 10% palladium on carbon (Degussa type) (7.75 g wetted with water 1:1 w:w) for 3.5 h. The reaction mixture was filtered then evaporated under reduced pressure and the residue was partitioned between dichoromethane (400 ml) and treated with saturated aqueous sodium hydrogen carbonate (400 ml, care CO₂). The organic phase was separated by hydrophobic frit and evaporated under reduced pressure to afford the title compound as a pair of diastereomers (15 g).
HPLC Rt=3.27 minutes (gradient 2); m/z [M+H]⁺=566
Intermediate 4 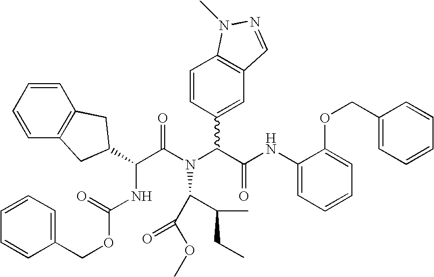
Methyl N-[(2R)-2-(2,3-Dihydro-1H-inden-2-yl)-2-({[(phenylmethyl)oxy]carbonyl}amino)acetyl]-N-[1-(1-methyl-1H-indazol-5-yl)-2-oxo-2-({2-[(phenylmethyl)oxy]phenyl}amino)ethyl]-D-alloisoleucinate

1-Methyl-1H-indazole-5-carbaldehyde (5.78 g, 34 mmol)) and (D)-alloisoleucine methyl ester hydrochloride (6.17 g, 34 mmol) in 2,2,2-trifluoroethanol (100 mL) were treated with triethylamine (4.74 mL, 34mmol) and the mixture was stood under nitrogen at room temperature for 18 h. (2R)-[(Benzyloxycarbonyl)amino](2,3-dihydro-1H-inden-2-yl)ethanoic acid (11.05 g, 34 mmol) and 2-benzyloxyphenylisonitrile (7.52 g, 36 mmol) were added and the mixture was stirred at room temperature under nitrogen for 3 days. The mixture was concentrated under reduced pressure then partitioned between ethyl acetate (750 mL) and water (500 mL). The aqueous phase was back-extracted with ethyl acetate (250 mL) and the combined organic extracts were washed with sat. sodium chloride (250 mL), dried over anhydrous magnesium sulphate, filtered and evaporated under reduced pressure to give the crude product (29.6 g). This was purified on a Redisep® silica column (330 g) eluted with 10-50% ethyl acetate in cyclohexane to afford 22.6 g of the title compound as a pair of diastereomers.
HPLC Rt=4.13 minutes (gradient 2); m/z [M+H]⁺=822.6

### EXAMPLE 1

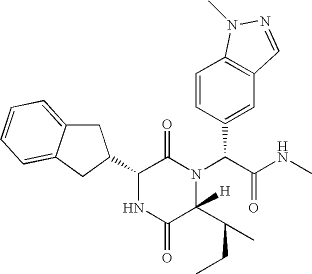 (2R)-2-{(3R,6R)-3-(2,3-Dihydro-1H-inden-2-yl)-6-[(1S)-1-methylpropyl]-2,5-dioxo-1-piperazinyl}-N-methyl-2-(1-methyl-1H-indazol-5-yl)ethanamide

2-{(3R,6R)-3-(2,3-dihydro-1H-inden-2-yl)-6-[(1S)-1-methylpropyl]-2,5-dioxo-1-piperazinyl}-N-(2-hydroxyphenyl)-2-(1-methyl-1H-indazol-5-yl)acetamide (intermediate 3) (0.5 g) and 1,1′-carbonyldiimidazole (0.23 g) were dissolved in dry dichloromethane (10 ml) and left to stand at room temperature under N₂ for 3 hours. A 2.0 M solution of methylamine in tetrahydrofuran (2.2 ml) was added and the reaction mixture was left to stand for 3 hours. The reaction mixture was evaporated in vacuo.

The residue was applied to a silica cartridge (35 g) and eluted with a gradient of ethyl acetate to 10% methanol in ethyl acetate. The required fractions were evaporated in vacuo and the residue was purified further using a SCX SPE cartridge (5 g), washed with methanol and the methanol was concentrated to afford (2R)-2-{(3R,6R)-3-(2,3-dihydro-1H-inden-2-yl)-6-[(1S)-1-methylpropyl]-2,5-dioxo-1-piperazinyl}-N-methyl-2-(1-methyl-1H-indazol-5-yl)ethanamide as a white solid.
HPLC Rt=2.9 minutes (gradient 1); m/z [M+H]⁺=488.

¹H NMR (CDCl₃) δ 7.99 (s, 1H), 7.79 (s, 1H), 7.47 (dd, 1H), 7.42 (d, 1H), 7.25-7.12 (m, 4H), 6.55 (d, 1H), 6.12 (q, 1H), 5.04 (s, 1H), 4.10 (s, 3H), 4.06 (dd, 1H), 3.96 (d, 1H), 3.22-3.05 (m, 3H), 2.97 (m, 1H), 2.85 (d, 3H), 2.76 (dd, 1H), 1.99 (m, 1H), 1.79 (m, 1H), 1.15 (m, 1H), 1.08 (d, 3H), 0.93 (t, 3H).

### EXAMPLE 1

2R)-2-{(3R,6R)-3-(2,3-Dihydro-1H-inden-2-yl)-6-(1S)-1-methylpropyl]-2,5-dioxo-1-piperazinyl}-N-methyl-2-(1-methyl-1H-indazol-5-yl)ethanamide

2-{(3R,6R)-3-(2,3-Dihydro-1H-inden-2-yl)-6-[(1S)-1-methylpropyl]-2,5-dioxo-1-piperazinyl}-N-(2-hydroxyphenyl)-2-(1-methyl-1H-indazol-5-yl)acetamide (15 g) and 1,1′-carbonyldiimidazole (6.88 g) were dissolved in dry dichloromethane (300 mL) under nitrogen and stirred for four hours at room temperature. A 2.0 M solution of methylamine in tetrahydrofuran (66.3 mL) was added over 10 minutes then the reaction mixture stirred for 30 minutes, the reaction mixture was then left to stand for 18 hours. The reaction mixture was diluted with dichloromethane (200 mL) and washed with 0.1 M HCl (400 mL). The organic extract was separated by hydrophobic frit and the aqueous extract was washed with further dichloromethane (200 ml). The combined organic extracts were concentrated in vacuo and the residue was applied to a redisep® silica cartridge (339 g) and eluted with a gradient of ethyl acetate to 10% methanol in ethyl acetate. The required fractions were evaporated in vacuo and the residue was purified further using a SCX-2 SPE cartridge (50 g) conditioning the cartridge with methanol and then loading and eluting the compound with methanol. On concentration of the relevant fractions this gave (2R)-2-{(3R,6R)-3-(2,3-dihydro-1H-inden-2-yl)-6-[(1S)-1-methylpropyl]-2,5-dioxo-1-piperazinyl}-N-methyl-2-(1-methyl-1H-indazol-5-yl)ethanamide (4.1 g, 32%) as a white solid.
HPLC Rt=2.9 minutes (gradient 1); m/z [M+H]⁺=488.

### EXAMPLE 2

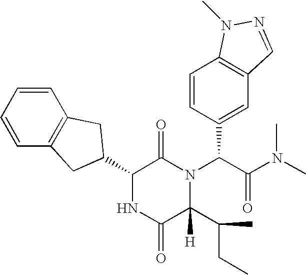 (2R)-2-{(3R,6R)-3-(2,3-Dihydro-1H-inden-2-yl)-6-[(1S)-1-methylpropyl]-2,5-dioxo-1-piperazinyl}-N,N-dimethyl-2-(1-methyl-1H-indazol-5-yl)ethanamide

The title compound was similarly prepared from intermediate 3 and dimethylamine.
HPLC Rt=3.0 minutes; m/z [M+H]⁺=502

¹H NMR (CDCl₃) δ 8.02 (s, 1H), 7.82 (s, 1H), 7.53-7.44 (m, 2H), 7.30-7.15 (m, 4H), 6.45 (s, 1H), 6.20 (d, 1H), 4.16-4.10 (m, 5H), 3.19-3.11 (m, 3H), 2.99-2.85 (m, 1H), 2.96 (s, 3H), 2.87 (s, 3H), 2.75 (dd, 1H), 1.50 (m, 1H), 1.05 (m, 1H), 0.78 (m, 1H), 0.60 (t, 3H), 0.39 (d, 3H).

### REFERENCE

1. V. Auwers; Lange; Chem. Ber.; 55; 1922; 1141, 1157.

2: Halley, Frank; Sava, Xavier. Synthesis of 5-cyanoindazole and 1-methyl and 1-aryl-5-cyanoindazoles. Synthetic Communications (1997), 27(7), 1199-1207.

Biological Activity

Examples 1 and 2 of the present invention were tested in all of the assays described below. Results for each of the compounds are shown in Table 1 below. The table also includes two compounds X and Y for comparison.

Assay 1

Determination of Antagonist Affinity at Human Oxytocin-1 Receptors using FLIPR

Cell Culture

Adherent Chinese Hamster Ovary (CHO) cells, stably expressing the recombinant human Oxytocin-1 (hOT) receptor, were maintained in culture in DMEM:F12 medium (Sigma, cat no D6421), supplemented with 10% heat inactivated foetal calf serum (Gibco/lnvitrogen, cat. no.01000-147), 2 mM L-glutamine (Gibco/lnvitrogen, cat. no. 25030-024) and 0.2 mg/ml G418 (Gibco/lnvitrogen, cat no. 10131-027). Cells were grown as monolayers under 95%:5% air:CO₂ at 37° C. and passaged every 3-4 days using TrypLE™ Express (Gibco/lnvitrogen, cat no. 12604-013).

Measurement of [Ca²⁺]₁ using the FLIPR™

CHO-hOT cells were seeded into black walled clear-base 384-well plates (Nunc) at a density of 10,000 cells per well in culture medium as described above and maintained overnight (95%:5% air:CO₂ at 37° C.). After removal of culture medium, cells were incubated for 1 h at 37° C. in Tyrode's medium (NaCl, 145 mM; KCl, 2.5 mM; HEPES, 10 mM; Glucose, 10 mM; MgCl₂, 1.2 mM; CaCl₂, 1.5 mM) containing probenacid (0.7 mg/ml), the cytoplasmic calcium indicator, Fluo-4 (4 uM; Teflabs, USA) and the quenching agent Brilliant Black (250 uM; Molecular Devices, UK). Cells were then incubated for an additional 30min at 37° C. with either buffer alone or buffer containing OT antagonist, before being placed into a FLIPR™ (Molecular Devices, UK) to monitor cell fluorescence (λₑₓ=488 nm, λ_{EM}=540 nm) before and after the addition of a submaximal concentration of oxytocin (EC80).

Data Analysis

Functional responses using FLIPR were analysed using Activity Base Version 5.0.10.

Assay 2

Oxytocin Binding Assay

Preparations

Membranes were prepared from CHO cells expressing human recombinant oxytocin receptors. The membrane preparation was frozen in aliquots at −70° C. until used.

The membrane preparation was frozen in aliquots at −70° C. until used.

Binding Assay Protocol

Membranes (˜50 ug) were incubated in 200 ul of assay buffer (50 mM Tris, 10 mM MgCl₂, and 0.1% bovine serum albumin, pH 7.5) containing ˜2.4 nM of [3H]-oxytocin in the absence (total binding) or presence (non-specific binding) of 1 uM unlabeled oxytocin and increasing concentrations of the compounds in Examples 1 and 2 or comparator compounds. Incubations were performed at room temperature for 60 minutes. The reactions were stopped with 3 ml of ice cold buffer and filtered through Whatman GF/C filter paper presoaked in 0.3% polyethylenimine. The filters were washed 4 times with 3 ml buffer using a Brandel cell harvester. The filters were counted in 3 ml Ready Safe scintillation fluid (Beckman).

Specific binding represented approximately 90% of total binding.

Data Analysis

IC₅₀ values were determined from competition binding experiments using non-linear regression analysis (GraphPad) and converted to Ki using the method of Cheng and Prusoff, 1974. Data are reported as mean values.

Assay 3

Determination of In Vitro Intrinsic Clearance in Microsomes

NADP regeneration buffer for use in incubations was prepared fresh on the assay day. It contained 7.8 mg glucose-6-phosphate (mono-sodium salt), 1.7 mg NADP and 6 Units glucose-6-phosphate dehydrogenase per 1 mL of 2% sodium bicarbonate. Microsomes (human, female; cynomolgus monkey, female; dog, female; rat, female) were prepared in pH 7.4 phosphate buffer and contained 0.625 mg protein/mL. Unless stated, all subsequent steps were performed by a Tecan Genesis 150/8 RSP. A 1.25 mM stock solution of the compounds was prepared in Acetonitrile/water (1:1). 25 ul of the 1.25 mM stock solution was added to 600 ul of Acetonitrile/water (1:1) to give a 50 uM solution. For each species, the 50 uM solutions (10 uL) were added to microsomes (790 uL) in a microplate (Porvair, 96 deepwell, square).

400 uL of the microsomal solution containing the compound was transferred to a microplate (Porvair, 96 deepwell, round) and was pre-warmed at 37° C. for five minutes prior to initiation of incubations. All incubations were initiated by addition of 100 uL of NADP regeneration system to the pre-warmed microsomes. The mixtures were incubated at 37° C. in a Techne heating block. Following 0, 3, 6, 12 and 30 minutes incubation, 20 uL aliquots were taken and added to 100 uL of acetonitrile containing internal standard.

For determination of the rate of metabolism, incubations were performed at a compound concentration of 0.5 uM and a protein concentration of 0.5 mg/mL. The concentration of solvent in the incubation was 0.5%.

Test compound concentrations were determined by LC/MS/MS; results were reported as analyte:internal standard peak area ratios.

The rate of disappearance was calculated by fitting a single exponential decay to the concentration-time curve using Excel and intrinsic clearance was calculated using the following formula:

$$Cli = \frac{[ rate ( 1 / min ) * 52.5 mg protein \text{/} g liver ]}{0.5 mg protein \text{/} mL}$$

Results

Examples 1 and 2 of the present invention and also two comparator compounds (Comparator compound X=(2R)-2-[(3R,6R)-3-(2,3-dihydro-1H-inden-2-yl)-6-isobutyl-2,5-dioxopiperazin-1-yl]-2-(1H-indazol-5-yl)-N,N-dimethylethanamide (Example 172 in WO 03/053443) and Comparator compound Y=(2R)-2-(2,4-difluorophenyl)-2-[(3R,6R)-3-(2,3-dihydro-1H-inden-2-yl)-6-isobutyl-2,5-dioxopiperazin-1-yl]-N,N-dimethylethanamide (Example 8 in WO 03/053443) were tested in the above assays, except comparator compound X was not tested in assays 1 and 2.

However, Comparator compound Y was tested in assays 1 and 2 and showed a similar potency to that exhibited by compounds 1 and 2 of the present invention, in fact each of these compounds exhibited fpki's of between 8.5 and 8.7 (Assay 1) and pKi's of between 9.9 and 10.4 (Assay 2).

However, the compounds of the present invention exhibited a surprising improvement in in vitro intrinsic clearance in microsomes (Assay 3) when compared with both of the comparator compounds X and Y.

TABLE 1

Assay 3 - Microsomal Cl
(ml/min/g)

| | Rat | Dog | Cyno | Human |
|---|---|---|---|---|
| Comparator | ++ | +++ | +++++ | +++++ |
| compound | | | | |
| X | | | | |
| Comparator | + | + | ++++ | +++ |
| compound | | | | |
| Y | | | | |
| Example 1 | + | + | ++ | + |
| Example 2 | + | + | ++ | + |

Key to Table 1
+ corresponds to 1-8 ml/min/mg
++ corresponds to 9-15 ml/min/mg
+++ corresponds to 16-20 ml/min/mg
++++ corresponds to 21-30 ml/min/mg
+++++ corresponds to >31 ml/min/mg

What is claimed is:

1. A compound of formula (1A), 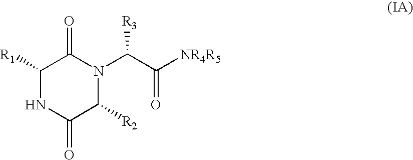
wherein R₁ is 2-indanyl, R₂ is 1-methylpropyl, R₃ is 1-methyl-indazol-5-yl, R₄ represents methyl and R₅ represents hydrogen or methyl, and pharmaceutically acceptable derivatives thereof.

2. A salt or solvate of formula (1A), according to claim 1, 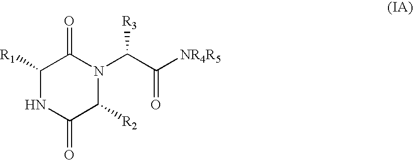
wherein R₁ is 2-indanyl, R₂ is 1-methylpropyl, R₃ is 1-methyl-indazol-5-yl, R₄ rep resents methyl and R₅ represents hydrogen or methyl.

3. A compound of formula (1A), according to claim 1, 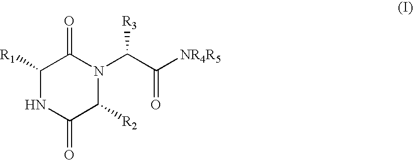
wherein R₁ is 2-indanyl, R₂ is 1-methylpropyl, R₃ is 1-methyl-indazol-5-yl, R₄ represents methyl and R₅ represents hydrogen and pharmaceutically acceptable derivatives thereof.

4. A compound according to claim 1 wherein R₂ is (1S)-1 -methylpropyl.

5. A compound according to claim 1 selected from:
(2R)-2-{(3R,6R)-3-(2,3-dihydro-1H-inden-2-yl)-6-[(1S)-1-methylpropyl]-2,5-dioxo-1 -piperazinyl}-N-methyl-2-(1-methyl-1H-indazol-5-yl)ethanamide, and pharmaceutically acceptable derivatives thereof.

6. A compound according to claim 1 selected from:
(2R)-2-{(3R,6R)-3-(2,3-dihydro-1H-inden-2-yl)-6-[(1S)-1 -methylpropyl]-2,5-dioxo-1 -piperazinyl}-N-methyl-2-(1 -methyl-1 H-indazol-5-yl)ethanamide, and
(2R)-2-{(3R,6R)-3-(2,3-dihydro-1H-inden-2-yl)-6-[(1S)-1-methylpropyl]-2,5-dioxo-1-piperazinyl}-N, N-dimethyl-2-(1-methyl-1 H-indazol-5-yl)ethanamide, and pharmaceutically acceptable derivatives thereof.

7. A pharmaceutical composition comprising a compound according to claim 1 and a pharmaceutically acceptable carrier.

8-10. (canceled)

11. A method of treating or preventing diseases or conditions mediated through the action of oxytocin which comprises administering to a mammal in need thereof of an effective amount of at least one chemical entity according to claim 1.

12. A method according to claim 11 wherein the disease or condition is selected from pre-term labour, dysmenorrhea, endometriosis and benign prostatic hyperplasia.

13. A process for the preparation of compounds of formula (I) or of formula (IA) as claimed in claim 1 respectively which comprises:
(a) reacting a compound of formula (II) 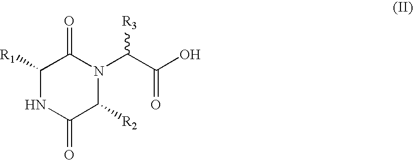
wherein R₁, R₂ and R₃ have the meanings defined in claim 1 and the chirality at R₃ is either R or S or a mixture thereof, or an activated derivative thereof, with the amine HNR₄R₅ wherein R₄ and R₅ have the meanings defined in claim 1 under standard conditions for preparing amides from a carboxylic acid or an activated derivative thereof and an amine, or
(b) reacting a compound of formula (III) 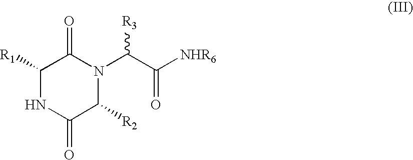
wherein R₁, R₂ and R₃ have the meanings defined in claim 1, and R₆ is 2-hydroxyphenyl, with 1,1′-carbonyldiimidazole or 1,1′-thiocarbonyldiimidazole in a suitable solvent and subsequent reaction of the product thus formed with amine HNR₄R₅ wherein R₄ and R₅ have the meanings defined claim 1.
